(19) United States
(12) Patent Application Publication
(10) Pub. No.: US 2016/0249988 A1
(43) Pub. Date: Sep. 1, 2016

(54) SURGICAL REFERENCING APPARATUS, SURGICAL NAVIGATION SYSTEM AND METHOD

(71) Applicant: Aesculap AG, Tuttlingen (DE)

(72) Inventors: Tobias Pfeifer, Waldmoessingen (DE); Holger Broers, Uplengen-Spols (DE); Sascha Beneke, Holtland-Nuecke (DE)

(21) Appl. No.: 15/149,790

(22) Filed: May 9, 2016

Related U.S. Application Data
(63) Continuation of application No. PCT/EP2014/074019, filed on Nov. 7, 2014.

(30) Foreign Application Priority Data
Nov. 11, 2013 (DE) 10 2013 112 375

(51) Int. Cl. A61B 34/20; A61B 90/00

(52) U.S. Cl. CPC A61B 34/20; A61B 90/39; A61B 2090/3991; A61B 2034/2051; A61B 1/04

(57) ABSTRACT

The invention relates to a surgical referencing apparatus, comprising a marking device having at least two or more surgical marker elements which are configured for reflecting and/or emitting radiation, wherein the marker elements form a marker element arrangement for defining a reference on the body of a patient, and comprising a fixing device for fixing the marking device in place on the body of the patient. In order to provide such a referencing apparatus that allows less invasiveness when fixing the referencing apparatus to the body of a patient, the invention proposes that the marker element arrangement be of non-rigid configuration, wherein two or more marker elements are variable in position relative to one another. Furthermore, the invention relates to a surgical navigation system and a method.

This application is a continuation of international application number PCT/EP2014/074019 filed on Nov. 7, 2014 and claims the benefit of German Application No. 10 2013 112 375.9 filed on Nov. 11, 2013, which are incorporated herein by reference in their entirety and for all purposes.

### BACKGROUND OF THE INVENTION

The invention relates to a surgical referencing apparatus, comprising a marking device having at least two or more surgical marker elements which are configured for reflecting and/or emitting radiation, wherein the marker elements form a marker element arrangement for defining a reference on the body of a patient, and comprising a fixing device for fixing the marking device in place on the body of the patient.

Furthermore, the invention relates to a surgical navigation system for tracking a surgical referencing apparatus in space, wherein the navigation system comprises a referencing apparatus comprising a marking device having at least two or more marker elements, a detection apparatus for detecting radiation emitted or reflected by the marker elements and for providing positional data related thereto, and a data processing apparatus for processing the positional data and for determining, on the basis thereof, the position and orientation of the referencing apparatus in space.

Furthermore, the invention relates to a method for tracking a surgical referencing apparatus in space using a surgical navigation system.

Referencing apparatuses of the type mentioned at the outset are known, these being in particular used in knee or hip prosthetics in order to facilitate for the surgeon the implantation of an artificial knee joint or an artificial hip joint. The referencing apparatus is fixed in place on a body part whose movement in space is to be tracked by way of the navigation system. Based on the positional data acquired by the navigation system when detecting radiation derived from the marker elements (in particular electromagnetic radiation and specifically infrared radiation), the data processing apparatus can make a calculation of the position and orientation of the marker element arrangement known to it from which it determines the position and orientation of the body part (in particular femur or tibia) that is to be tracked in space.

In order to obtain a reliable reference, the marking devices are configured in the form of what are known as “rigid bodies” which have a fixedly predetermined relative arrangement of the marker elements to each other that is known to the navigation system. For achieving a stable reference, the referencing apparatus is connected via the fixing device to the femur or to the tibia by screwing, commonly by way of a bone screw, in some cases placed bicortically, and is thereby securely fixed thereto. However, this leads to an intervention of considerable invasiveness and some surgeons and patients have reservations about this. In order to reduce the invasiveness, it is known to fix rigid bodies to a cuff that is placed around the femur or the tibia; however, in practice this has proven to adversely affect the accuracy of the reference.

It is an object of the present invention to provide a referencing apparatus of the generic kind and to provide a navigation system of the generic kind that allows less invasiveness when fixing the referencing apparatus to the body of a patient.

### SUMMARY OF THE INVENTION

In accordance with the invention, this object is achieved in a referencing apparatus of the kind mentioned at the outset by the marker element arrangement being of non-rigid configuration, wherein two or more marker elements are variable in position relative to one another.

In the referencing apparatus constructed in accordance with the invention, the possibility exists for two or more marker elements to be variable in position relative to one another and to be movable relative to one another. The geometry of the marker element arrangement is allowed to change. This abandons the traditional rigid body concept of defining a reference which, in order to be reliable, requires a rigid marker element arrangement in which the individual marker elements are rigidly connected together and cannot move relative to each other. The invention incorporates the idea that in a non-rigid marker element arrangement, a mobility of the marker elements relative to one other can be allowed, provided that the relative movement of the marker elements during the intended use of the referencing apparatus with the navigation system is generally small relative to the absolute movement of the marker element arrangement, which is based on a movement of the referencing apparatus when the body part is moved.

With the referencing apparatus constructed in accordance with the invention, it is in particular possible for the body part to be marked in a manner that is less invasive than is the case when the referencing apparatus is fixed via, for example, a bone screw. Preferably, it is in particular possible for the marker elements to be fixed to the patient's body via a cuff or the like. In contrast to conventional rigid bodies that are likewise fixed to the body part by use of a cuff, the advantage of the referencing apparatus constructed in accordance with the invention, however, relies on the relative mobility of the marker elements. In conventional referencing apparatuses comprising rigid marker element arrangements, it cannot be determined whether a movement of the marking device in space is based on a movement of the body part or on a movement of the marking device relative to the body part due to non-rigid fixing. In contrast, by the use of the referencing apparatus constructed in accordance with the invention, this can be determined under the assumption that the relative mobility of the marker elements with respect to each other is smaller than the absolute movement of the marker element arrangement in space due to the movement of a body part.

Preferably, the marker elements are capable of being positioned separately from each other, in particular capable of being individually fixed in place on the body of the patient or on a carrier body of the fixing device. In this way, the referencing apparatus proves to be particularly versatile. The marker elements can be positioned in such a way that the detection apparatus of the navigation system can get as good a view of them as possible and that as accurate a reference as possible can be achieved, even when the marker elements are variable in position relative to each other.

It proves advantageous for the marker elements to be variable in position relative to each other in pairs.

Provision may also be made for part of the marker elements to be non-variable in position relative to each other, whereas at least one further marker element is variable in position relative to the first-named marker elements.

Preferably, the marker elements are variable in position relative to each other in groups, wherein a group of marker elements comprises at least two marker elements and wherein two or more groups of marker elements are provided.

It is particularly advantageous for the fixing device to be configured such that the marking device is capable of being fixed in place on the body of the patient in a non-invasive manner. As used herein, the term “non-invasive” is in particular taken to mean that the marking device is capable of being fixed in place on the body of the patient without an incision, in particular without utilizing a separate incision only for fixing the marking device in place, and/or without the use of a bone anchoring element.

Preferably, the fixing device comprises a fixing element on one or more marker elements. Each marker element can have a fixing element of its own associated with it, or two or more marker elements can have a common fixing element associated with them. The fixing elements of the marker elements may be identical or different. Via the fixing element, the marker elements can be fixed directly to the patient's body or indirectly via for example the carrier body described below.

In an advantageous embodiment of the referencing apparatus constructed in accordance with the invention, at least one fixing element comprises an adhesive layer. Via the adhesive layer, the marking device can be adhesively fixed directly to the body of the patient or to a carrier body that is in turn fixed to the body of the patient.

Alternatively or in addition, provision may be made for at least one fixing element to be a loop tape or a hook tape for cooperating with a hook tape and a loop tape respectively on a carrier body of the fixing device. By the cooperation between a loop tape and a hook tape, a hook-and-loop connection-type fixing can be formed for fixing the marker element to the carrier body. Each marker element can have a separate loop tape or hook tape associated with it, or a plurality of marker elements have a common loop tape or hook tape associated with them.

Alternatively or in addition, provision may be made for at least one fixing element to be or to comprise a surgical bone anchoring element. At least one fixing element may be a bone anchoring element, such as a bone screw or a Kirschner wire. The different fixing elements are preferably a loop tape or a hook tape, a suction cup, or a fixing element comprising an adhesive layer. A marker element that is fixed to a bone via a bone anchoring element can form a particularly stable reference on the body of the patient for improving accuracy. This reference can be considered by the navigation system to be more trustworthy when it comes to distinguishing between an absolute movement of the marker element arrangement and a relative movement of the marker elements with respect to each other for determining the change in the position and orientation of the marker element arrangement.

It is advantageous for the fixing device to comprise or to form a carrier body that is associated with a plurality of the marker elements, said carrier body having the marker elements fixed or fixable in place thereon. Via the carrier body, a plurality of marker elements can be fixed in common to the body of the patient.

The carrier body is preferably variable in shape, whereby it can easily adjust to the anatomy of the patient.

It is advantageous for the marker elements to be capable of being positioned at and in particular on the carrier body in a manner separate from one another, whereby the referencing apparatus is rendered highly versatile.

It is advantageous for at least one marker element to be capable of being removably fixable in place on the carrier body, for example via a fixing element that comprises a releasable adhesive layer or a suction cup or via a hook-and-loop connection having fixing elements in the form of a loop tape and a hook tape. By the marker element being capable of being removably fixable in place on the carrier body, it can be repositioned. By way of example, this allows the accuracy of the reference formed by the marker element arrangement to be enhanced by, for example, the geometry and/or the size of the marker element arrangement being changed in such a way that it makes it easier for the navigation system to distinguish between the relative movement of the marker elements with respect to each other and the absolute movement of the referencing apparatus.

Provision may be made for at least one marker element to be non-removably fixed in place on the carrier body by, for example, being sewed, adhesively bonded or thermally welded thereto.

The carrier body preferably comprises a hook tape or a loop tape for cooperating with a loop tape and a hook tape respectively on one or more marker elements. One or more marker elements can thereby be removably connected to the carrier body via a hook-and-loop connection, as has already been mentioned.

In an advantageous embodiment of the referencing apparatus constructed in accordance with the invention, it proves advantageous for the carrier body to be or to comprise a textile composite. The textile composite can easily adjust to the patient's anatomy, whereby the referencing apparatus is provided with a high versatility. In particular, it provides an easy way of non-invasively fixing the marking device in place on the patient's body.

In an advantageous embodiment, the carrier body is or comprises a bandage. Said bandage is for example a strap-on bandage, preferably variable in length, that can be easily adjusted to fit various sizes of body parts.

In a further advantageous embodiment of the referencing apparatus, the carrier body advantageously is or comprises a tubular or stocking body. In particular, the carrier body can have an annular shape and can be slipped onto the body part in the manner of a tubular sleeve or stocking and can thereby be fixed in place thereon. The carrier body has a deformable, for example rubber-elastic, configuration for adjustment to the patient.

In a configuration of the referencing apparatus that is structurally simple, it is advantageous for the carrier body to be capable of being adhesively connected to the patient's body. By configuring the carrier body in the form of an adhesive patch, the referencing apparatus can be of particularly simple configuration and can be fixed to the patient's body in a particularly simple manner.

In the last-mentioned advantageous embodiment in particular, it is advantageous if the carrier body is of sheet-like configuration.

In a further advantageous embodiment of the referencing apparatus constructed in accordance with the invention, the carrier body comprises or forms at least one hollow space and at least one connecting element for a suction and/or a pressure conduit, wherein the at least one hollow space is capable of having a negative or positive pressure applied thereto. The at least one hollow space can have a negative or positive pressure applied thereto via the at least one connecting element, whereby the dimensional stability of the carrier body can be enhanced. By way of example, such configuration is employed for a carrier body in the form of a bandage, a cuff or a tubular or stocking body, wherein the carrier body can be fixed in place on the patient's body in an easy-to-handle manner by change in shape. In a subsequent application of negative or positive pressure, the dimensional stability of the carrier body can be enhanced. It is thereby possible, for example, to reduce the mobility of the marker elements relative to each other while achieving a more reliable fixing of the carrier body to the patient's body.

Provision may be made for the fixing device to comprise a fixing element on one or more marker elements and/or at least one fixing element arranged on the carrier body, which fixing element is or comprises a suction cup. The suction cup provides an easy-to-handle way for the marker element to be removably fixed in place (on the carrier body or on the patient's body directly) or for the carrier body to be removably fixed in place on the patient's body.

The carrier body can, on the side thereof facing towards the referencing apparatus during intended use, comprise friction coefficient-enhancing elements with which as high a friction as possible can be achieved between the carrier body and the body of the patient. By way of example, dot-shaped or strip-shaped friction coefficient-enhancing elements are provided, in particular made of a silicone material.

In order to achieve a configuration that is simple in structure, it is advantageous for the marker elements to be identical.

As mentioned previously, the invention further relates to a surgical navigation system. The object postulated at the outset is achieved, in accordance with the invention, in a surgical navigation system of the generic kind by the referencing apparatus being a referencing apparatus of the kind described above.

In the navigation system constructed in accordance with the invention, it is in particular possible to determine whether a movement of the marker elements is attributable to a movement of the body part having the referencing apparatus fixed thereto or to a movement of the marker elements within the marker element arrangement in which the marker element can move relative to one another. With respect to the relative mobility of the marker elements to each other within the marker element arrangement, it is assumed by the data processing apparatus that, when the navigation system is used, this will be generally smaller than an absolute movement of the referencing apparatus in space.

The advantages that can be achieved in the context of the description of the referencing apparatus constructed in accordance with the invention can also be achieved using the navigation system; therefore, in this respect, reference may be had to what has been stated above, in order to avoid repetition. Furthermore, reference is made to what has been explained in the context of advantageous embodiments of the referencing apparatus and of the advantages that can be achieved thereby.

It is advantageous for the data processing apparatus to be configured and programmed such that it determines the geometry of the marker element arrangement on the basis of the positional data when the referencing apparatus is moved in space. The position of the individual marker elements relative to each other, or the relative orientation thereof, defines the geometry of the marker element arrangement. The data processing apparatus can, in a sense, “automatically” determine same without it having to be known to the data processing apparatus as is the case with conventional referencing apparatuses. In particular, this also affords the possibility of automatically identifying a referencing apparatus without previous knowledge of its presence.

Provision may be made for outputting to the user, on an indication unit of the navigation system, an indication to move the patient's body part including the referencing apparatus affixed thereto in space. Based on the positional data, the data processing apparatus can analyse, determine and store the number and relative orientation of the marker elements and hence the geometry of the marker element arrangement in order to make the marker element arrangement the basis for tracking the referencing apparatus in space.

Preferably, the data processing apparatus may be configured and programmed such that by analysis of the positional data when tracking the referencing apparatus in space, it also automatically determines a change in the marker element arrangement. The stored geometry can be replaced with the newly determined geometry of the marker element arrangement if required.

Advantageously, the data processing apparatus is configured and programmed such that it determines what own mobility the marker elements have within the marker element arrangement and such that it stores the respective own mobility in a storage unit or updates an already stored own mobility and uses this as a basis for a subsequent determination of the change in the position and orientation of the referencing apparatus in space. This is preferably realized in the initial determination of the geometry of the marker element arrangement, as described above, or subsequent thereto, for example before the actual intervention. However, it is also possible for this to be carried out during the intervention when tracking the referencing apparatus in space. In particular, the respective own mobility of a marker element can be continuously monitored and updated by the data processing apparatus.

In the present case, the own mobility of a marker element is considered to be the capability thereof for movement within the marker element arrangement relative to the other marker elements. Such a movement can for example be attributed to the deformation of the body part which has the referencing apparatus fixed in place thereon, to a movement or deformation of the carrier body which has the marker element fixed in place thereon, or to a movement of the marker element relative to the carrier body when the marker element is connected to same in non-rigid relationship.

By knowing the own mobility of the marker elements, the data processing apparatus, when tracking the referencing apparatus, can compensate for a movement of the marker elements that leads to a change in the geometry of the marker element arrangement. Accordingly, the data processing apparatus can determine whether a marker element has moved within its own mobility and hence, in a sense, “within acceptable limits”. Within these limits, the data processing apparatus considers the position of the marker element within the marker element arrangement as being correct.

Preferably, the data processing apparatus is configured and programmed such that in the determination of the own mobility of the marker elements, it takes into account the amount by which and/or the spatial direction in which the respective marker element is movable within the marker element arrangement relative to the other marker elements of the marking device. By way of example, the data processing apparatus defines an ellipsoid for the respective mobility of a marker element having three axes that are pairwise perpendicular to one another, their amount in each case indicating the capability of a marker element to move along the corresponding axes within the marker element arrangement.

It is advantageous for the data processing apparatus to be configured and programmed such that in a determination of the change in the position and orientation of the referencing apparatus in space, it gives different weighting to positional data of marker elements depending on the magnitude of the respective own mobility thereof, wherein marker elements that have low mobility of their own are given a higher weighting than marker elements having a larger mobility of their own. The data processing apparatus can consider marker elements having a relatively low mobility of their own to be more trustworthy than marker elements having a relatively high mobility of their own. This allows the data processing apparatus to determine a measure of the certainty or accuracy of positional data that are to be attributed to a marker element. Accordingly, when tracking the referencing apparatus, thereby determining the marker element arrangement, the data processing apparatus can weight positional data of a trustworthy marker element more strongly than those of a less trustworthy marker element. A measure of the certainty and accuracy with which the marker element arrangement is recognized can thereby also be determined.

It is advantageous for the data processing apparatus to be configured and programmed such that, taking into account the own mobilities of the marker elements, it estimates the accuracy with which it is able to determine a change in the position and orientation of the referencing apparatus in space, and such that, if there is insufficient accuracy, the data processing apparatus outputs on an indication unit of the navigation system an indication proposing to change the position of at least one marker element or to add at least one further marker element to the marker element arrangement. With the goal of achieving as accurate a reference as possible with the referencing apparatus, the data processing apparatus can preferably output one or more indications on the indication unit (for example a display screen) already in the initial recognition and identification of the marker element arrangement and of the own mobilities of the marker elements. By changing the position of one or more marker elements or by adding at least one marker element, the measure of the accuracy with which the referencing apparatus can be tracked in space can be enhanced.

By way of example, the data processing apparatus is configured and programmed such that it provides an indication related to the change in the position of a marker element whose own mobility exceeds a threshold mobility.

Advantageously, the data processing apparatus is configured and programmed such that in the determination of the change in the position and orientation of the referencing apparatus in space, it determines whether a movement of a respective marker element within the marker element arrangement exceeds a threshold movement. When the referencing apparatus is tracked in space, the data processing apparatus can determine, on the basis of the positional data, whether the marker element moves within the marker element arrangement relative to the other marker elements in a manner that exceeds a threshold movement. By way of example, a check is made as to whether the movement of the marker element lies within its own mobility, in particular within the above-mentioned mobility ellipsoid of the marker element.

Advantageously, the data processing apparatus is configured and programmed such that if the answer is affirmative, it does not weight the positional data of the respective marker element as strongly as the positional data of other marker elements having smaller movements within the marker element arrangement in the determination of the change in the position and orientation of the referencing apparatus in space. As has already been mentioned, this affords the possibility of considering the marker elements as being of different trustworthiness when tracking the referencing apparatus. Marker elements of less movement within the marker element arrangement can be given a stronger weighting for tracking the referencing apparatus than marker elements whose movement exceeds the threshold movement.

Advantageously, the data processing apparatus is configured and programmed such that it ignores the positional data of the respective marker element in the determination of the change in the position and orientation of the referencing apparatus in space, provided that the data processing apparatus can unambiguously determine the position and orientation of the referencing apparatus in space based on the non-ignored positional data of the remaining marker elements. The marker element whose movement exceeds the threshold movement can be ignored in the determination of the marker element arrangement provided that unambiguous determination of same is possible on the basis of the positional data of the remaining marker elements. In a sense, only those marker elements the data processing apparatus considers as “valid” are made use of in order to determine the marker element arrangement.

Correspondingly, provision may be made for a marker element outside the field of view of the detection apparatus to be ignored, i.e. when the data processing apparatus determines that there exist no positional data regarding the corresponding marker element. The data processing apparatus can then proceed to identify the marker element arrangement on the basis of the positional data of the remaining marker elements provided that this can be done unambiguously.

Provision may be made for the data processing apparatus to know the position of a body's own structure or of an axis, surface or plane in space defined thereby. By way of example, the position in space of a plane, such as the pelvic inlet plane, or the position in space of a body axis, for example the femoral mechanical axis or the tibial axis, is known. This makes it possible to make use of a reference of the referencing apparatus that is fixed to the body. When the referencing apparatus is tracked in space, a target geometry such as the body's own structure or axis, surface or plane can be tracked therewith.

It is in particular advantageous for the data processing apparatus to be configured and programmed such that, when the position of the referencing apparatus relative to a body's own structure or an axis, surface or plane of the patient defined thereby is known, it estimates the accuracy with which it is able to determine a change in the position of the body's own structure, axis, surface or plane in space, and such that, if there is insufficient accuracy, the data processing apparatus outputs on an indication unit of the navigation system an indication proposing to change the position of at least one marker element or to add at least one further marker element to the marker element arrangement. This affords the possibility of enhancing the accuracy with regard to the body's own structure, axis, surface or plane. Tracking the body's own structure, axis, surface or plane as accurately as possible on the basis of the referencing apparatus is of advantage for knowledge of the anatomical conditions of the patient, for example in hip or knee surgery.

It is advantageous for the data processing apparatus to be configured and programmed such that it outputs on an indication unit of the navigation system, for example on a display screen and preferably before the actual intervention, an indication to the user regarding the number and/or the position of the marker elements to be used in the arrangement thereof on the body of the patient, in particular regarding the distance of the marker elements from one another and/or their arrangement relative to one another, and/or an indication regarding how to fix the marker elements to the body of the patient via the fixing device. By way of example, the navigation system can propose the number of marker elements, their distance from each other, their relative position, the type of body part and/or the type of fixing, thereby providing guidance and assistance to the surgeon in the arrangement of the referencing apparatus. In particular, this is done with the goal of achieving as accurate a reference as possible with the referencing apparatus.

It proves advantageous for the navigation system to comprise a storage unit which stores patterns for marker element arrangements regarding the number and/or the relative orientation of the marker elements, their position of fixing in place on the body of the patient and/or regarding how the marker elements are fixed to the body of the patient via the fixing device, and for the data processing apparatus to be configured and programmed such that it outputs the indication taking into account at least one of the patterns read from the storage unit. The user can have an indication output to him or her on the basis of the patterns or “templates”. The surgeon can thereby be particularly specifically guided and assisted in the positioning and/or in the fixing of the marker elements on the basis of experience-based values and/or prior knowledge.

As mentioned at the outset, the invention further relates to a method. A method in accordance with the invention with which the object postulated at the outset can also be achieved is provided for tracking a surgical referencing apparatus in space with the use of a surgical navigation system of the kind mentioned above, said navigation system comprising a referencing apparatus of the kind mentioned above, wherein a detection apparatus of the navigation system detects radiation emitted or reflected by the marker elements and provides positional data related thereto and wherein a data processing apparatus of the navigation system processes the positional data for determining the position and orientation of the referencing apparatus in space, wherein the data processing apparatus determines a change in the position and orientation of the referencing apparatus in space.

The advantages that can be achieved in the context of the referencing apparatus constructed in accordance with the invention and of the navigation system constructed in accordance with the invention can be achieved using said method. Therefore, reference is made to what has been stated above, also in the context of the respective embodiments, in order to avoid repetition. Advantageous embodiments of the referencing apparatus and the navigation system can be used to form advantageous exemplary embodiments of the method; therefore, in this respect as well, reference is made to what has been stated above.

The following description of preferred embodiments of the invention, taken in conjunction with the drawings, serves to explain the invention in greater detail.

### BRIEF DESCRIPTION OF THE DRAWINGS

FIG. 1 is a perspective view of a navigation system constructed in accordance with the invention, shown as being applied on a patient, comprising an advantageous embodiment of a referencing apparatus constructed in accordance with the invention, wherein the navigation system is configured for carrying out an advantageous exemplary embodiment of the method in accordance with the invention;

FIG. 2 is a representation at an enlarged scale of detail A of FIG. 1;

FIG. 3 is a perspective representation of the referencing apparatus of the navigation system of FIG. 1 (in opened state) and also illustrates a leg of the patient;

FIG. 4 shows a marker element of the referencing apparatus prior to it being fixed in place on a carrier body of the referencing apparatus (partially represented);

FIG. 5 shows the marker element as being fixed to the carrier body (partially represented);

FIG. 6 is a representation corresponding to FIG. 3, associated with a second advantageous embodiment of a referencing apparatus constructed in accordance with the invention;

FIG. 7 is a representation corresponding to FIG. 3, associated with a third advantageous embodiment of a referencing apparatus constructed in accordance with the invention;

FIG. 8 shows, in side view, the referencing apparatus of FIG. 7 (partially represented);

FIG. 9 is a representation corresponding to FIG. 3, associated with a fourth advantageous embodiment of a referencing apparatus constructed in accordance with the invention;

FIG. 10 is a side view of the referencing apparatus of FIG. 9 (partially represented);

FIG. 11 is a schematic side view of a fifth advantageous embodiment of a referencing apparatus constructed in accordance with the invention;

FIG. 12 is a schematic side view of a sixth advantageous embodiment of a referencing apparatus constructed in accordance with the invention; and

FIG. 13 is a schematic top view of a carrier body of a referencing apparatus constructed in accordance with the invention.

### DETAILED DESCRIPTION

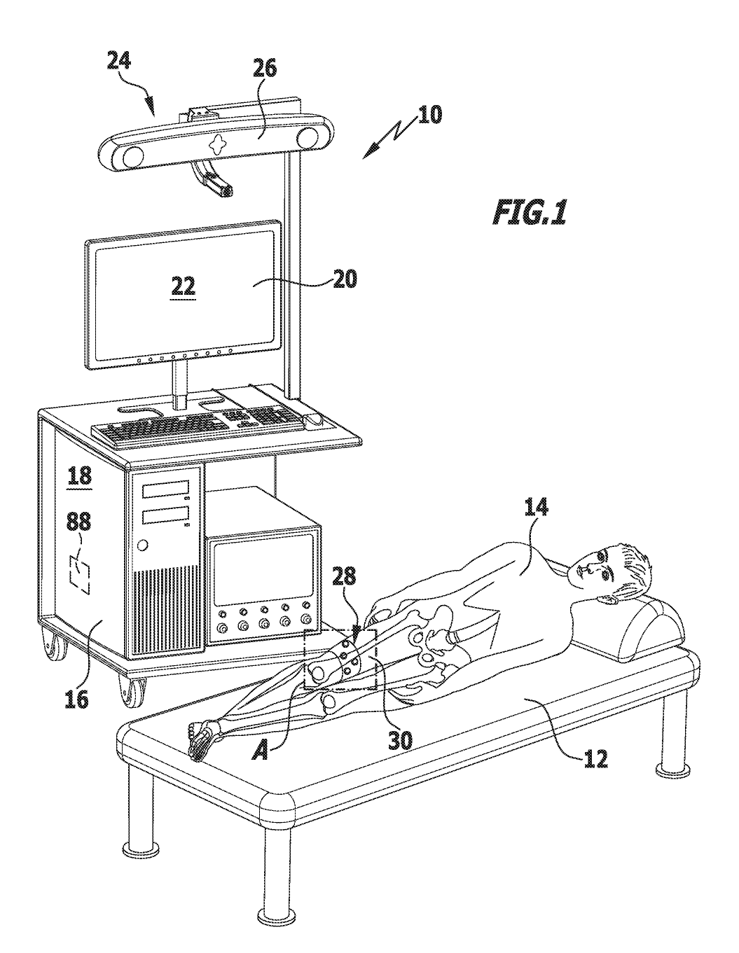

FIG. 1 shows a perspective representation of an advantageous embodiment of a navigation system constructed in accordance with the invention, designated by the reference character 10, with a patient 14 placed in a lying position on an operating table 12.

The navigation system 10 comprises a data processing apparatus 16 in the form of a computer 18 and, connected thereto, an indication unit 20 in the form of a display screen 22. Furthermore, the navigation system 10 comprises a detection apparatus 24 which comprises in particular a stereoscopic camera 26. The stereoscopic camera 26 allows electromagnetic radiation, in particular infrared radiation, to be detected and a corresponding signal to be transmitted to the data processing apparatus 16. The stereoscopic camera 26 is also capable of emitting electromagnetic radiation, which is reflected by a referencing apparatus of the navigation system 10 and is then detected again by the stereoscopic camera 26.

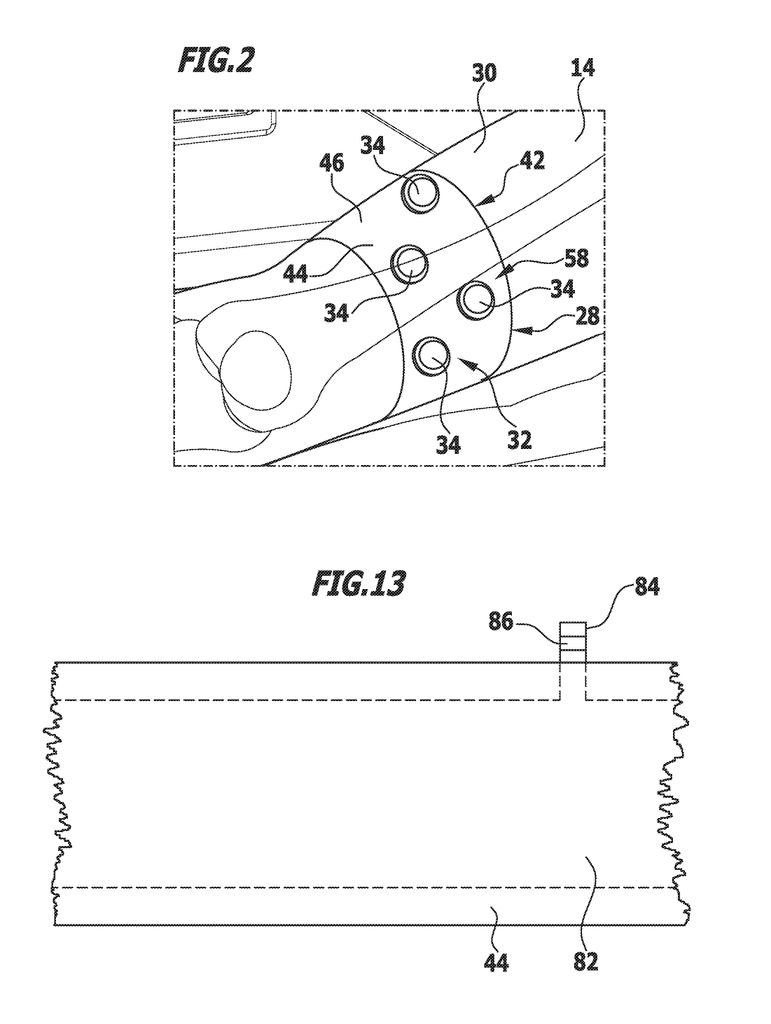

FIG. 1 illustrates an advantageous embodiment of a referencing apparatus constructed in accordance with the invention, designated by the reference character 28; this is shown enlarged in FIG. 2 and in more detail in FIG. 3. In the present case, the referencing apparatus 28 is fixed to a body part of the patient 14, namely to the thigh 30, for example in the vicinity of the knee. When the thigh 30 moves in space, for example during surgical procedures on the knee or hip, the referencing apparatus 28 also moves in space. It defines a reference on the basis of which the navigation system 10 can determine a movement of the referencing apparatus 28, and hence of the thigh 30, in space. In particular, the position and/or the orientation of the referencing apparatus 28 and the thigh 30 can be determined by the computer 18, as will be explained subsequently.

The referencing apparatus 28 comprises a surgical marking device 32 which has a plurality of marker elements 34. The marker elements 34 are of identical configuration. In the present case, a total of four marker elements 34 are present.

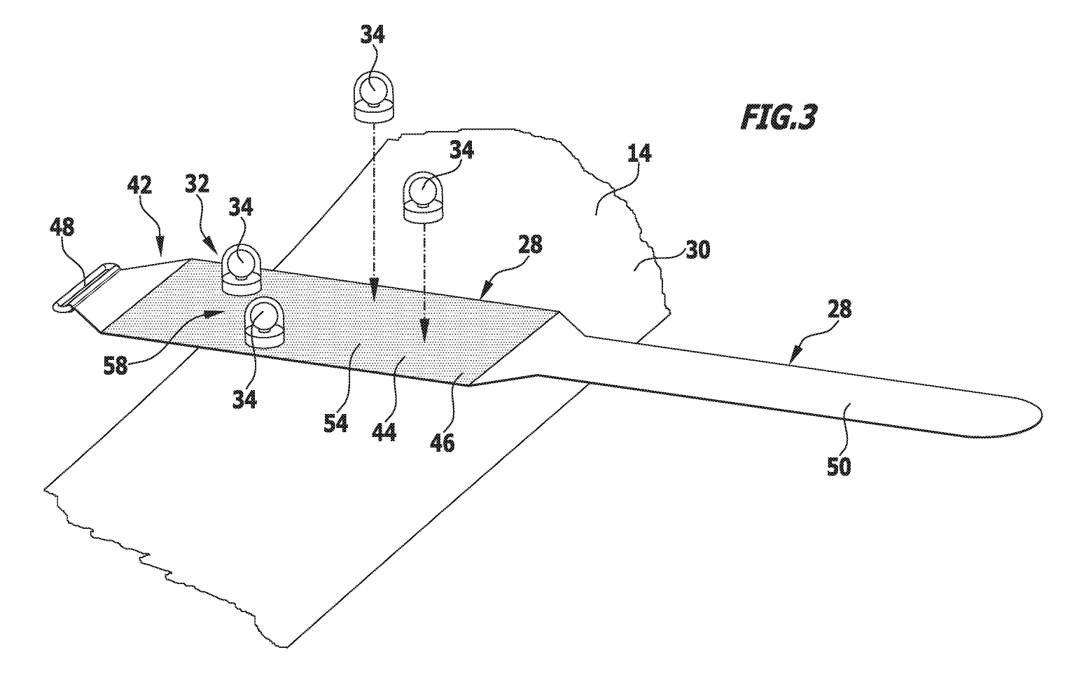

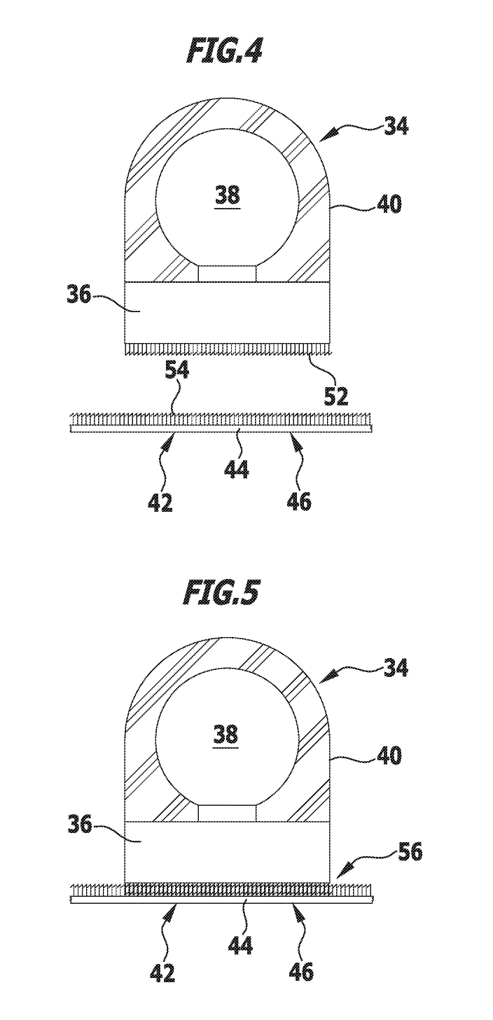

The construction of the marker elements 34 can be seen from the schematic representations of FIGS. 3 to 5. Each marker element 34 comprises a base element 36. The base element 36 forms a base or socket for a reflecting element 38 that reflects the electromagnetic radiation from the stereoscopic camera 26. The marker element 34 has a hood-shaped protective element 40 for protecting the reflecting element 38, said protective element 34 covering the reflecting element 38 and the base element 36 and being transparent to the electromagnetic radiation of the stereoscopic camera 26.

The referencing apparatus 28 comprises a fixing device 42 for fixing the marking device 32 to the patient 14. The fixing device 42 comprises a carrier body 44 which is capable of being removably fixed to the patient 14. In the present case, the carrier body 44 is associated with all of the marker elements 34, these being capable of being fixed in placed thereon as will be explained below.

In the referencing apparatus 28, the carrier body 44 is a bandage 46. By way of example, the bandage 46 may be fabricated from a textile composite or a rubber-elastic material. The bandage 46 can be transferred from an open, elongated state (FIG. 3) to a ring-shaped state in which it goes around the thigh 30 in the shape of a ring.

The bandage 46 is variable in length and can be fixed in itself. To this end, it has on a first side thereof an eyelet 48 which can have a bandage section 50 located on the opposite side of the bandage 46 passed therethrough, whereby the bandage 46 can be pulled tight. Alternatively, fixing the bandage 46 in itself can be accomplished for example by way of a buckle, snap fasteners or through hook-and-loop closure (not shown).

The bandage 46 is deformable, whereby it can adjust very well to the anatomy of the patient 14. By the additional possibility of adjusting the length of the bandage by way of the bandage section 50 and the eyelet 48, the bandage 46 and hence the referencing apparatus 28 proves to be particularly versatile.

The carrier body 44 can have friction coefficient-increasing elements, for example studs or ribs of a silicone material, on the side thereof that faces towards the patient 14 during intended use. This gives the possibility of securely fixing the bandage 46 to the patient 14, in addition to fixing the bandage 46 in itself.

The fixing device 42 further comprises cooperating fixing elements 52, 54.

Each marker element 34 has a fixing element 52 associated therewith, said fixing element 52 being fixed to the respective base element 36. The fixing elements 52 are loop or hook tapes. The fixing element 54 is a hook tape or a loop tape on the carrier body 44 and can cooperate with the loop or hook tape on the respective marker element 34.

As a result, the fixing elements 52, 54 form a hook-and-loop connection 56. Via the hook-and-loop connection 56, the marker elements 34 can be removably fixed in place on the carrier body 44. Each marker element 34 can be individually fixed in place on the carrier body 44 and removed again therefrom, whereby the marker elements are capable of being positioned separately from one another and, via the fixing device 42, capable of being fixed in place on the patient 14 separately from one another.

In combination with each other, the marker elements 34 form a marker element arrangement 58. The marker element arrangement 58 is variable in respect of its geometry because the marker elements 34 are not rigidly connected together but are capable of being individually fixed to the carrier body 44. The geometry of the marker element arrangement 58 can also vary by the bandage 46 deforming.

Because the marker elements 34 are individually fixed in place on the bandage 46, relative movement of the marker elements 34 relative to each other and hence a change in the geometry of the marker element arrangement 58 can occur.

In this regard, it is possible for the marker elements 34 to have different mobilities. One or more marker element(s) 34 can then have a higher mobility within the marker element arrangement 58 than one or more other marker elements 34. Conversely, it is possible for one or more marker element(s) 34 to have a lower mobility within the marker element arrangement 58 than one or more other marker elements 34.

The geometry of the marker element arrangement 58 can also be varied by repositioning at least one marker element 34 on the carrier body 44. Based on the removable connection formed by the hook-and-loop connection 56, the user can remove at least one marker element 34 from the carrier body 44 and refix it in place thereon in a different position.

The marker element arrangement 58 can also be varied by a marker element 34 being removed altogether or by at least one further marker element 34 being added to the marking device 32.

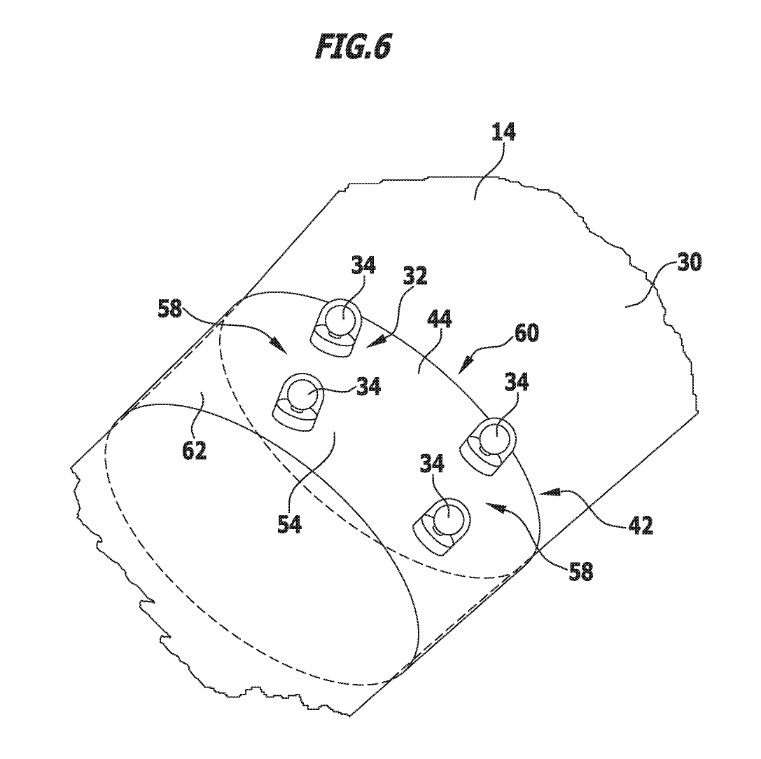

Further advantageous embodiments of the referencing apparatus constructed in accordance with the invention will be discussed hereinafter with reference to FIGS. 6 to 12. The same reference characters are used to denote features or components of the respective referencing apparatus that are identical or functionally equivalent.

FIG. 6 shows a referencing apparatus 60 having four marker elements 34. The fixing device 42 comprises a carrier body 44 which is configured in the form of an annular tubular body 62 (or stocking body 62) that is closed in itself.

The tubular body 62 is for example fabricated as a textile composite structure or from a rubber-elastic material. In particular, the tubular body 62 is deformable to adjust to the anatomy of the patient 14. Here, too, friction coefficient-enhancing elements may be provided on the side that faces towards the patient 14. The side facing away from the patient 14 has arranged thereon the fixing element 54 for cooperation with the fixing elements 52 (not shown). Accordingly, the referencing apparatus 60, too, allows the marker elements 34 to be individually and removably connected to the tubular body 62 via a hook-and-loop connection.

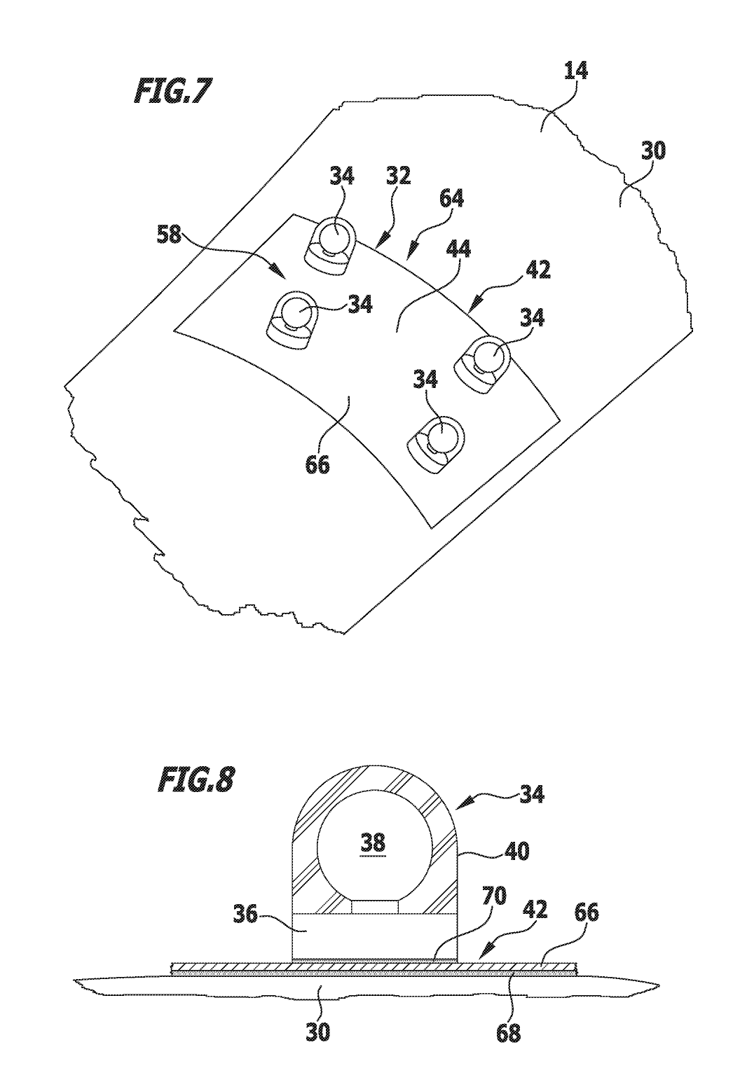

FIGS. 7 and 8 show a referencing apparatus, designated by the reference character 64, likewise comprising four marker elements 34. The fixing device 42 comprises the carrier body 44. The carrier body 44 is configured in the form of an adhesive patch 66. The fixing device 42 comprises an adhesive layer 68 on the adhesive patch 66 via which the latter can be removably attached to the patient 14, in particular to the thigh 30.

The fixing device 42 further comprises a fixing element 70, likewise configured in the form of an adhesive layer, on each marker element 34. This allows the marker elements 34 to be adhesively fixed to the adhesive patch 66, positioning them individually. Preferably, the marker elements 34 can be removed from the adhesive patch 66.

As an alternative to the adhesive layer, a hook-and-loop connection 56 can be employed for fixing the marker elements 34 to the adhesive patch 66.

The adhesive patch 66 is deformable, whereby it can adjust particularly well to the anatomy of the patient 14.

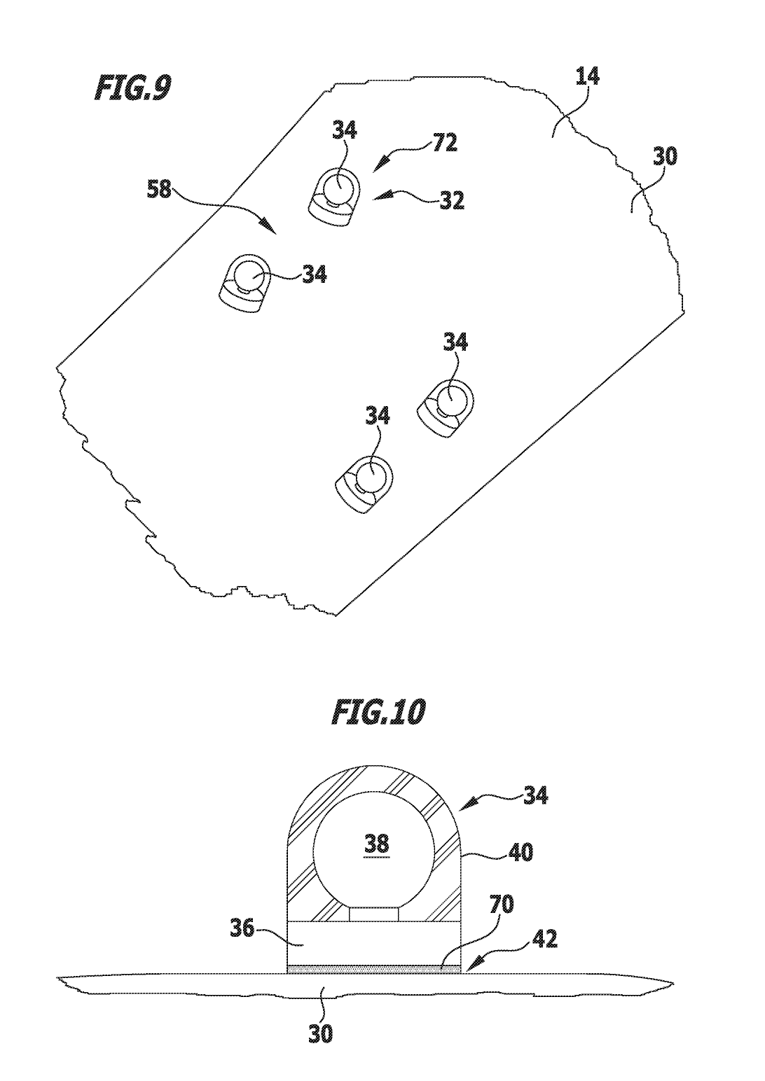

FIGS. 9 and 10 show a referencing apparatus, designated by the reference character 72, which likewise comprises four marker elements 34. In the referencing apparatus 72, a common carrier body for the marker elements 34 is omitted. Instead, these can be individually fixed to the patient 14, in particular to the thigh 30. To this end, the fixing device 42 comprises on each marker element 34 the fixing element 70, configured in the form of an adhesive layer.

Each marker element 34 can be individually and adhesively fixed to the patient 14. Based on the capability of the thigh 30 for deformation, the marker elements 34 are also movable relative to each other.

Of course, provision may be made for a plurality of marker elements 34 to be adhesively attached to the patient 14 via a common adhesive layer 70.

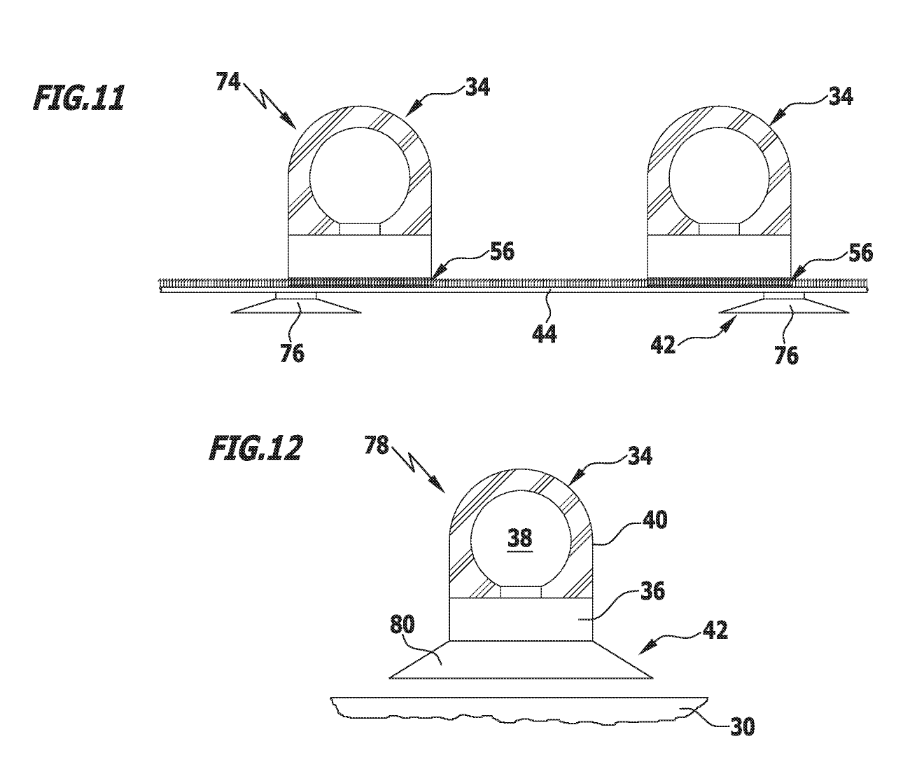

FIG. 11 shows a referencing apparatus, designated by the reference character 74, of which two marker elements 34 are illustratively shown. The marker elements 34 are connected to the carrier body 44 (for example the bandage 46, the tubular body 62 or the adhesive patch 66) via the hook-and-loop connection 56; however, they could also be fixed in place thereon via the adhesive layer 70.

In the referencing apparatus 74, the fixing device 42 comprises fixing elements 76 that are fixed in place on the carrier body 44 on the side thereof facing towards the patient 14. The fixing elements 76 are configured in the form of suction cups. Via the suction cups 76, the carrier body 44 can be removably fixed in place on the patient 14 in an easy-to-handle manner.

Of a referencing apparatus 78, partially represented in FIG. 12, only one marker element 34 is illustrated. However, the referencing apparatus 78 has more, for example four, marker elements 34.

The fixing device 42 comprises a fixing element 80. The fixing element 80 is configured in the form of a suction cup which is arranged on the side of the base element 36 that faces towards the patient. The suction cup 80 provides an easy-to-handle way for removably connecting the marker element 34 to the patient 14, in particular to the thigh 30.

In particular, fixing elements that are configured in the form of suction cups can also be employed in variants of the referencing apparatuses 64 and 74.

FIG. 13 shows, in schematic top view, a portion of a carrier body 44 in an advantageous embodiment of a referencing apparatus constructed in accordance with the invention. The carrier body 44 is for example the bandage 46 or the tubular body 62.

The carrier body 44 forms a hollow space 82 for a gas, such as air in particular. A connecting element 84 on the carrier body 44 permits a negative or positive pressure to be applied to the hollow space 82, whereby the dimensional stability of the carrier body 44 can be enhanced. The connecting element 84 can have a valve element 86 provided thereon in order to seal off the hollow space 82 in a gastight manner.

The navigation system 10 can comprise a pressure application device, such as a suction pump and/or a pressure pump. The pressure application device can be connected to the connecting element 84 via a suction and/or pressure conduit, not illustrated in the drawing, thereby enabling the carrier body 44 to be deflated or inflated.

By way of example, provision may be made for the carrier body 44 to be fixed to the patient 14 without it having pressure applied thereto. Subsequently, the carrier body 44 can have a negative or positive pressure applied thereto in order to improve its fixation to the patient 14. At the same time, the dimensional stability of the carrier body 44 can be enhanced. The deformability of the carrier body 44 is thereby reduced, whereby the mobility of the marker elements 34 relative to one another can also be reduced.

The referencing apparatuses 60, 64, 72, 74 and/or 78 can be employed in the navigation system 10 in lieu of or in addition to the referencing apparatus 28.

With all of the referencing apparatuses 28, 60, 64, 72, 74 and 78, the marker element arrangement 58 formed in each case by the marker elements 34 is non-rigid. In particular, it can vary based on the deformability of the bandage 46, the tubular body 62, the adhesive patch 66 and the deformability of the thigh 30 and, as previously mentioned, by repositioning, removing or adding one or more marker elements 34.

All of the referencing apparatuses 28, 60, 64, 72, 74 and 78 have the great advantage of being capable of being fixed to the patient 14 in a non-invasive manner, whereby additional trauma to the patient 14 can be avoided, in particular in procedures of hip or knee surgery.

In the following, the tracking of the referencing apparatus 28 in space for determining a change in the position and orientation of the thigh 30 will be discussed, wherein the following statements also apply to the case where the referencing apparatus 60, 64, 72, 74 and/or 78 is used.

In the referencing apparatus 28, although the possibility exists that the marker elements 34 move relative to each other, thereby changing the geometry of the marker element arrangement 58, the computer 18 is still able, on the basis of positional data from the detection apparatus 24, to determine the change in position and orientation of the referencing apparatus 28 and hence of the thigh 30. In particular, this is realized by taking into account the assumption that although the relative movement of the marker elements 34 with respect to each other is possible, it is generally smaller than an absolute movement of the marker element arrangement 58 in space. A sufficient determination of the position and orientation of the thigh 30 can thereby still be made in a reliable manner despite the non-invasive fixing of the referencing apparatus 28.

In the navigation system 10, in particular, the possibility exists that the computer 18 determines in a sense “automatically” the geometry of the marker element arrangement 58 when the referencing apparatus 28 is moved in space. For example, prior to the actual intervention, the user can be presented with an indication on the display screen 22 to move the thigh 30 including the bandage 46 attached thereto. The computer 18 analyses the positional data of the detection apparatus 24 and is capable of determining the geometry of the marker element arrangement 58 without previous knowledge of the presence of the marker elements 34, their number and relative arrangement.

The geometry of the marker element arrangement 58 can be stored in a storage unit 88 of the computer.

When the thigh 30 is moved, the computer 18 can further determine, on the basis of the positional data, the own mobilities of the marker elements 34. Here, the own mobility of a marker element 34 can be considered in particular to be its capability of moving within the marker element arrangement 58 relative to the other marker elements 34. The movement of a marker element occurs for example as a result of a deformation of the bandage 46 in itself or based on a deformation of the thigh 30 or based on the non-rigid fixation of the marker element 34 to the bandage 46.

For the own mobility determination, the computer 18 can determine the extent to which the marker element 34 can move and/or the spatial direction in which it can move. By way of example, the computer 18 defines an ellipsoid of mobility for each marker element 34 and, for tracking the referencing apparatus 28 in space, it assumes that the marker element 34 will normally move within the range of said mobility ellipsoid relative to a stationary arrangement of the marker element arrangement 58.

The own mobilities of the marker elements 34 can also be stored in the storage unit 88. These are taken as a basis for tracking the referencing apparatus 28 in space, as is the geometry of the marker element arrangement 58.

Taking into account the geometry of the marker element arrangement 58 and the own mobilities of the marker elements 34, the computer 18 can make an estimate of the accuracy with which a reliable tracking of the referencing apparatus 28 in space is possible. If the computer 18 finds that this does not appear to be the case, then it can indicate this fact to the user via, for example, the display screen 22. Said indication can comprise the proposal of repositioning at least one marker element 34 or adding at least one marker element 34. In the case of such repositioning or addition of at least one marker element 34, the navigation system can store the marker element arrangement 58 anew and take this as the basis for the further tracking of the referencing apparatus 28.

For further assistance to the user when the referencing apparatus 28 is being arranged and fixed on the patient 14 already, resort can be had to experience-based knowledge that is stored in the navigation system 10.

By way of example, the storage unit 88 has stored therein patterns (known as “templates”) for marker element arrangements with regard to the number and/or relative arrangement of the marker elements 34 and/or the positioning thereof on the carrier body 44 for forming a predeterminable geometry of the marker element arrangement 58. Furthermore, a respective pattern can have stored therein information pertinent to the type or arrangement of the referencing apparatus 28 on the patient 14 or information pertinent to the advantages of a respective referencing apparatus 28, 60, 64, 72, 74 or 78.

Based on prior knowledge stored in such a pattern, the computer 18 is able to provide guidance to the surgeon via the display screen 22, assisting him or her in fixing the referencing apparatus 28 (or any one of the other referencing apparatuses), in particular the marker elements 34 thereof, to the patient 14 in such a way that the navigation system 10 can be expected to reliably recognize the geometry of the marker element arrangement 58.

When tracking the referencing apparatus 28 in space, the computer 18 can, upon analysis of the positional data, compensate for movements of the marker elements 34 within the marker element arrangement 58 by taking into account the own mobilities thereof. In particular, the possibility exists for the positional data to be weighted differently depending on the magnitude of the respective own mobility. By way of example, positional data of a marker element 34 that has a relatively small own mobility can be considered to be more trustworthy than positional data of a marker element 34 whose own mobility is relatively large. This enables the computer 18 to establish a measure of the reliability and of the determinability in the tracking of the referencing apparatus 28 in space.

In the tracking of the referencing apparatus 28 in space, in order to determine the change in position and/or orientation thereof, the detection apparatus 24 establishes data sets in successive time intervals (for example at a frequency of 25 Hz). These are analysed by the computer 18 with regard to the positional data of the marker elements 34. By way of example, the computer 18 can determine whether the movement of a marker element 34 within the marker element arrangement 58 exceeds a threshold movement. By way of example, the previously determined own mobility of the marker element 34 can be used as the threshold condition.

If this is the case, the computer 18 can determine that positional data of the corresponding marker element 34 be given a lower weighting in order to identify the referencing apparatus 28 in space. Where required, the positional data of said marker element 34 may even be ignored completely during an ongoing determination. This is in particular possible in those instances where unambiguous determination of the position and orientation in space of the marker element arrangement 58, and hence of the referencing apparatus 28, is possible on the basis of the positional data of the remaining marker elements 34.

Correspondingly, the positional data of marker elements whose movement within the marker element arrangement 58 is below a threshold movement can be used as being of particular trustworthiness. Such a marker element 34 can then be used for example as a reference marker element the positional data of which are used as a basis for deriving an absolute movement of the marker element arrangement 58 in space.

The computer 18 can monitor and, if required, update the own mobilities of the marker elements 34 during the tracking of the referencing apparatus 28 so that these can be used as a basis for further calculations.

Correspondingly, during the tracking of the referencing apparatus, it is also possible to present the user with an indication output on the display screen 22 to reposition at least one marker element 34 or to add at least one marker element 34, with the goal of achieving as accurate a reference as possible.

What is claimed is:

1. Surgical referencing apparatus, comprising:
a marking device having at least two or more surgical marker elements which are configured for at least one of reflecting and emitting radiation, the marker elements forming a marker element arrangement for defining a reference on a body of a patient, and
a fixing device for fixing the marking device in place on the body of the patient,
wherein:
the marker element arrangement is of non-rigid configuration, and
the at least two or more marker elements are variable in position relative to one another.

2. Referencing apparatus in accordance with claim 1, wherein the marker elements are positionable separately from each other, in particular individually fixable in place on the body of the patient or on a carrier body of the fixing device.

3. Referencing apparatus in accordance with claim 1, wherein the marker elements are variable in position relative to each other in groups, wherein a group of marker elements comprises at least two marker elements and wherein two or more groups of marker elements are provided.

4. Referencing apparatus in accordance with claim 1, wherein the fixing device is configured such that the marking device is fixable in place on the body of the patient in a non-invasive manner.

5. Referencing apparatus in accordance with claim 1, wherein the fixing device comprises or forms a carrier body that is associated with a plurality of the marker elements, said carrier body having the marker elements fixed or fixable in place thereon.

6. Referencing apparatus in accordance with claim 5, wherein the carrier body is variable in shape.

7. Referencing apparatus in accordance with claim 5, wherein the marker elements are positionable on the carrier body in a manner separate from one another.

8. Referencing apparatus in accordance with claim 5, wherein the at least one marker element is removably fixable in place on the carrier body.

9. Referencing apparatus in accordance with claim 5, wherein the carrier body comprises a hook tape or a loop tape for cooperating with a loop tape and a hook tape respectively on one or more marker elements.

10. Referencing apparatus in accordance with claim 5, wherein the carrier body is or comprises a textile composite.

11. Referencing apparatus in accordance with claim 5, wherein the carrier body is or comprises a bandage or wherein the carrier body is or comprises a tubular or stocking body.

12. Surgical navigation system for tracking a surgical referencing apparatus in space, comprising:
a referencing apparatus comprising:
a marking device having at least two or more surgical marker elements which are configured for at least one of reflecting and emitting radiation, the marker elements forming a marker element arrangement for defining a reference on a body of a patient, and
a fixing device for fixing the marking device in place on the body of the patient,
wherein the marker element arrangement is of non-rigid configuration, and the at least two or more marker elements are variable in position relative to one another;
a detection apparatus for detecting radiation emitted or reflected by the marker elements and for providing positional data related thereto; and
a data processing apparatus for processing the positional data and for determining, on the basis thereof, the position and orientation of the referencing apparatus in space.

13. Navigation system in accordance with claim 12, wherein the data processing apparatus is configured and programmed such that it determines what own mobility the marker elements have within the marker element arrangement and such that the data processing apparatus stores the respective own mobility in a storage unit or updates an already stored own mobility and uses this as a basis for a subsequent determination of the change in the position and orientation of the referencing apparatus in space.

14. Navigation system in accordance with claim 13, wherein the data processing apparatus is configured and programmed such that in the determination of the own mobility, it takes into account the amount by which and/or the spatial direction in which the respective marker element is movable within the marker element arrangement relative to the other marker elements of the marking device.

15. Navigation system in accordance with claim 13, wherein the data processing apparatus is configured and programmed such that in a determination of the change in the position and orientation of the referencing apparatus in space, it gives different weighting to positional data of marker elements depending on the magnitude of the respective own mobility thereof, wherein marker elements that have low mobility of their own are given a higher weighting than marker elements having a larger mobility of their own.

16. Navigation system in accordance with claim 13, wherein the data processing apparatus is configured and programmed such that, taking into account the own mobilities of the marker elements, it estimates the accuracy with which it is able to determine a change in the position and orientation of the referencing apparatus in space, and such that, if there is insufficient accuracy, the data processing apparatus outputs on an indication unit of the navigation system an indication proposing to change the position of at least one marker element or to add at least one further marker element to the marker element arrangement.

17. Navigation system in accordance with claim 13, wherein the data processing apparatus is configured and programmed such that in the determination of the change in the position and orientation of the referencing apparatus in space, it determines whether a movement of a respective marker element within the marker element arrangement exceeds a threshold movement.

18. Navigation system in accordance with claim 17, wherein the data processing apparatus is configured and programmed such that if the answer is affirmative, it does not weight the positional data of the respective marker element as strongly as the positional data of other marker elements having smaller movements within the marker element arrangement in the determination of the change in the position and orientation of the referencing apparatus in space.

19. Navigation system in accordance with claim 17, wherein the data processing apparatus is configured and programmed such that it ignores the positional data of the respective marker element in the determination of the change in the position and orientation of the referencing apparatus in space, provided that the data processing apparatus can unambiguously determine the position and orientation of the referencing apparatus in space based on the non-ignored positional data of the remaining marker elements.

20. Method for tracking a surgical referencing apparatus in space with the use of a surgical navigation system, the navigation system comprising:
a referencing apparatus comprising:
a marking device having at least two or more surgical marker elements which are configured for at least one of reflecting and emitting radiation, the marker elements forming a marker element arrangement for defining a reference on a body of a patient, and
a fixing device for fixing the marking device in place on the body of the patient,
wherein the marker element arrangement is of non-rigid configuration, and the at least two or more marker elements are variable in position relative to one another;
a detection apparatus; and
a data processing apparatus;
the method comprising:
detecting, by means of the detection apparatus, radiation emitted or reflected by the marker elements;
providing of positional data related to the marker elements by the detection apparatus; and
processing the positional data via the data processing apparatus to determine a position and orientation of the referencing apparatus in space;
wherein the data processing apparatus determines a change in the position and orientation of the referencing apparatus in space.
